(19) United States
(12) Patent Application Publication
(10) Pub. No.: US 2005/0054669 A1
(43) Pub. Date: Mar. 10, 2005

(54) Process for the synthesis of zolpidem

(76) Inventors: Yatendra Kumar, Gurgaon (IN); Mohan Prasad, Gurgaon (IN); Asok Nath, Gurgaon (IN)

Correspondence Address: Ranbaxy Inc.;Suite 2100, 600 College Road East, Princeton, NJ, 08540, US

(21) Appl. No.: 10/909,468

(22) Filed: Aug. 2, 2004

(30) Foreign Application Priority Data
Jul. 31, 2003 (IN) 951/DEL/2003

(51) Int. Cl. C07D 471/04; A61K 31/4745

(52) U.S. Cl. 514/303; 546/119

(57) ABSTRACT

The present invention relates to processes for the preparation of zolpidem of Formula V as shown in the accompanying drawings or pharmaceutically acceptable salts thereof from N,N-dimethyl-3-(4-methyl)benzoyl propionamide of Formula II. The process includes (a) reacting N,N-dimethyl-3-(4-methyl)benzoyl propionamide of Formula II with bromine to get the bromo amide of Formula III; (b) condensing the bromo amide of Formula III with 2-amino-5-methylpyridine of Formula IV to get the zolpidem base of Formula V; and (c) converting zolpidem base of Formula V to its hemitartarate salt of Formula VII.

### FIELD OF THE INVENTION

The present invention relates to processes for the preparation of zolpidem of Formula V, as shown in the accompanying drawings, or pharmaceutically acceptable salts thereof from N,N-dimethyl-3-(4-methyl)benzoyl propionamide of Formula II.

### BACKGROUND OF THE INVENTION

Chemically, zolpidem hemitartrate is N,N,6-trimethyl-2-(4-methylphenyl)-imidazo[1,2-a]pyridine-3-acetamide L-(+)-hemitartrate of Formula VII, as shown in the accompanying drawings.

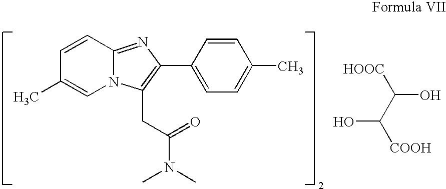

Zolpidem is disclosed in European Patent No. 50,563, which is an equivalent of U.S. Pat. No. 4,382,938. The pharmacological profile of this compound is characterized by a strong hypnotic effect, together with weak anticonvulsant and muscle-relaxant properties, selectivity for benzodiazepine receptors with the biochemical characteristics, and regional distribution of the benzodiazepine one subtype. While zolpidem is a hypnotic agent with a chemical structure unrelated to benzodiazepines, barbiturates, or other drugs with known hypnotic properties, it interacts with gamma-aminobutyric acid (GABA)-benzodiazepine receptor complex and shares some of the pharmacological properties of the benzodiazepines. The selective binding of zolpidem on the omega-1 receptor may explain the relative absence of myorelaxant and anticonvulsant effects in animal studies. Zolpidem shows both high affinity and selectivity toward non-benzodiazepine-2 receptors, which results in improved activity and/or fewer side effects for the treatment of anxiety, sleep disorders, and convulsions.

Zolpidem tartrate is sold in the United States under the brand name of Ambien® by Sanofi Synthelabo, Inc. and is approved for the short-term treatment of insomnia. According to the labeling for Ambien, zolpidem has been shown to decrease sleep latency and increase the duration of sleep. Zolpidem is available in 5 mg and 10 mg tablets for oral administration. The Ambien® tablet includes as inactive ingredients: hydroxypropyl methylcellulose, lactose, magnesium stearate, microcrystalline cellulose, polyethylene glycol, sodium starch glycolate, and titanium dioxide; the 5-mg tablet also contains FD&C Red No. 40, iron oxide colorant, and polysorbate 80.

Indian patent application 782/DEL/2000 relates to processes for the preparation of N,N-dimethyl-3-(4-methyl)benzoyl propionamide of Formula II, which is a key intermediate in the synthesis of zolpidem.

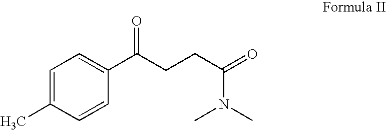

A general method for the synthesis of 2-phenylimidazo[1,2-a]pyridine of Formula I, wherein R₁, R₂, X, Y, Z and n are as defined in Table 1, as described in the accompanying drawing, is reported in J. Med. Chem., 40, 3109-3118 (1997). Preparation of zolpidem is, however, not discussed in this article. The reaction conditions employed therein are stringent and require higher temperature.

TABLE I

| | |
|---|---|
| | Formula I |
| 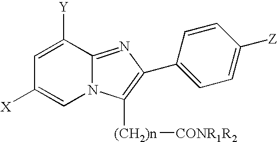 | |

| Compd | X | Y | Z | R₁ | R₂ | n |
|---|---|---|---|---|---|---|
| 1 | H | H | H | C₂H₅ | C₂H₅ | 0 |
| 2 | Cl | H | Cl | C₂H₅ | C₂H₅ | 0 |
| 3 | Cl | H | Cl | C₃H₇ | C₃H₇ | 0 |
| 4 | Cl | H | Cl | -(CH₂)₄- | | 0 |
| 5 | H | H | H | C₃H₇ | C₃H₇ | 1 |
| 6 | Cl | H | H | C₃H₇ | C₃H₇ | 1 |
| 7 | Br | H | H | C₃H₇ | C₃H₇ | 1 |
| 8 | I | H | H | C₃H₇ | C₃H₇ | 1 |
| 9 | CH₃ | H | H | C₃H₇ | C₃H₇ | 1 |
| 10 | CH₃O | H | H | C₃H₇ | C₃H₇ | 1 |
| 11 | NO₂ | H | H | C₃H₇ | C₃H₇ | 1 |
| 12 | Br | H | Cl | C₃H₇ | C₃H₇ | 1 |
| 13 | Cl | H | Cl | -(CH₂)₄- | | 1 |
| 14 | Cl | H | Cl | -(CH₂)₅- | | 1 |
| 15 | Cl | Cl | H | C₃H₇ | C₃H₇ | 1 |
| 16 | Cl | Cl | Cl | C₃H₇ | C₃H₇ | 1 |
| 17 | Br | Br | Cl | C₃H₇ | C₃H₇ | 1 |
| 18 | CF₃ | Cl | Cl | C₃H₇ | C₃H₇ | 1 |
| 19 | Br | CH₃ | H | C₃H₇ | C₃H₇ | 1 |
| 20 | CH₃ | Br | H | C₃H₇ | C₃H₇ | 1 |
| 21 | Cl | H | H | C₃H₇ | C₃H₇ | 2 |

### SUMMARY OF THE INVENTION

In one general aspect, there is provided a process for the preparation of zolpidem hemitartarate of Formula VII. The process steps include
a. reacting N,N-dimethyl-3-(4-methyl)benzoyl propionamide of Formula II with bromine to get the bromo amide of Formula III;
b. condensing the bromo amide of Formula III with 2-amino-5-methylpyridine of Formula IV to get the zolpidem base of Formula V; and
c. converting zolpidem base of Formula V to its hemitartarate salt of Formula VII.

Embodiments of the process may include one or more of the following features. For example, step a) may be carried out in an organic solvent. The organic solvent may be chlorinated hydrocarbon, ether, and mixtures thereof. For example chloroform, methylene chloride, dichloroethane, dibromoethane, carbon tetrachloride, tetrahydrofuran, diethyl ether, 1,4-dioxane, diisopropyl ether and mixtures thereof. In particular, the organic solvent may be chloroform.

The process, step b) may be performed in the presence of an organic solvent. The organic solvent may be acetone, diisopropyl ether, dimethylacetamide, dimethylformamide, toluene, methanol, isopropanol, and mixtures thereof. In particular, the organic solvent may be acetone.

In another general aspect, there is provided a pharmaceutical composition that includes the zolpidem hemitartarate of Formula VII made by the process that includes the following steps:
a. reacting N,N-dimethyl-3-(4-methyl)benzoyl propionamide of Formula II with bromine to get the bromo amide of Formula III;
b. condensing the bromo amide of Formula III with 2-amino-5-methylpyridine of Formula IV to get the zolpidem base of Formula V; and
c. converting zolpidem base of Formula V to its hemitartarate salt of Formula VII.

Embodiments of the pharmaceutical composition may include one or more of the following features or those described above. For example, the pharmaceutical composition may further include one or more pharmaceutically acceptable inactive ingredients.

In another general aspect, there is provided a method of treating insomnia. The method includes administering a pharmaceutical composition that includes the zolpidem hemitartarate of Formula VII made by the process that includes the following steps:
a. reacting N,N-dimethyl-3-(4-methyl)benzoyl propionamide of Formula II with bromine to get the bromo amide of Formula III;
b. condensing the bromo amide of Formula III with 2-amino-5-methylpyridine of Formula IV to get the zolpidem base of Formula V; and
c. converting zolpidem base of Formula V to its hemitartarate salt of Formula VII.

Embodiments of the method of treating may include one or more of the following features or those described above. For example, the pharmaceutical composition may further include one or more pharmaceutically acceptable inactive ingredients.

The details of one or more embodiments of the inventions are set forth in the description below. Other features, objects and advantages of the inventions will be apparent from the description and claims.

### DETAILED DESCRIPTION OF THE INVENTION

Herein is provided an improved and efficient method for the preparation of zolpidem of Formula V or its pharmaceutically acceptable salt of Formula VII (as shown in the accompanying drawings). The process advantageously provides benefits with respect to economics and convenience to operate at a commercial scale.

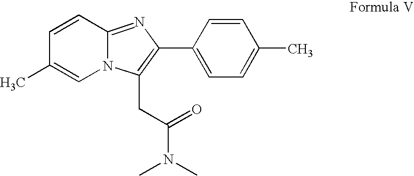

The general process described herein includes the following steps:

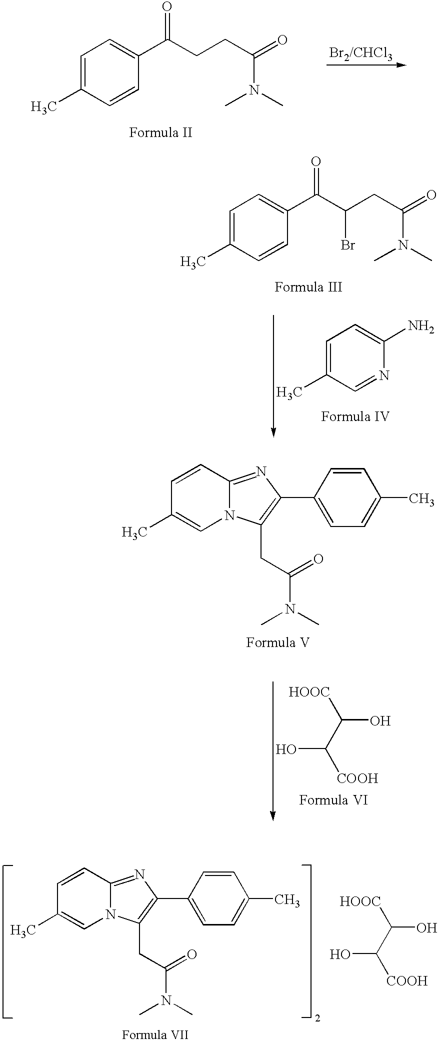

More particularly, herein is provided a process for the preparation of zolpidem of Formula V or its hemitartarate salt, the process including the following steps:
a. reacting N,N-dimethyl-3-(4-methyl)benzoyl propionamide of Formula II with bromine to get bromo amide of Formula III; 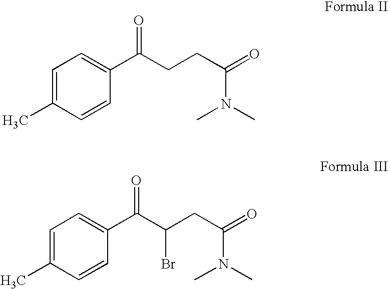
b. condensing the bromo amide of Formula III with 2-amino-5-methylpyridine of Formula IV to get zolpidem base of Formula V; and 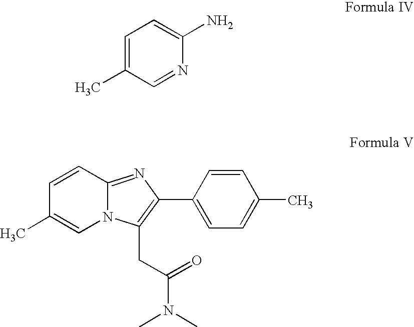
c. converting zolpidem base of Formula V to its hemitartarate salt of Formula VII.

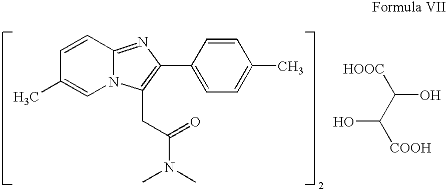

The starting material of the above process, N,N-dimethyl-3-(4-methyl)benzoyl propionamide of Formula II, which is a key intermediate in the synthesis of zolpidem, can be prepared as per Indian patent application 782/DEL/2000.

The intermediate, Formula II, is treated with bromine to get the 2-bromo amide of Formula III. The reaction is performed in presence of a suitable organic solvent. Suitable organic solvents include chlorinated hydrocarbon, ether and mixtures thereof. The organic solvent can be, for example, chloroform, methylene chloride, dichloroethane, dibromoethane, carbon tetrachloride, tetrahydrofuran, diethyl ether, diisopropyl ether, 1,4-dioxane and mixtures thereof. The reaction can be carried out at a temperature range of about 10 to about 100° C. for about 30 minutes to about 5 hours. In some particular embodiments, the reaction is carried out at a temperature from about 55-60° C. for about 1 hour.

The 2-bromo amide intermediate is refluxed with 2-amino-5-methylpyridine of Formula IV as shown in the accompanying drawing, to get zolpidem base of Formula V. The reaction is performed in the presence of an organic solvent. For example acetone, tetrahydrofuran, dimethylformamide, dimethylacetamide, toluene, diisopropyl ether and mixtures thereof. The reaction can be carried out at a temperature range of about 50 to about 100° C. for about 1 to about 50 hours. In some particular embodiments, the reaction is carried out at a temperature from about 50-80° C. for about 10 to 20 hours.

Zolpidem base of Formula V was treated with L-(+)-tartaric acid of Formula VI in methanol get zolpidem hemitartarate of formula VII as shown in the accompanying drawing.

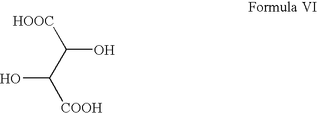

Particular embodiments are described below by way of examples to illustrate the process of this invention. However, these do not limit the scope of the present invention.

### Preparation of Zoldipem Hemitartarate

### EXAMPLE

Step a) Preparation of N,N-dimethyl-2-bromo-3-(4-methyl)benzoyl Propionamide (Formula III)

To a stirred solution of N,N-dimethyl-3-(4-methyl)benzoyl propionamide (140 gm) of Formula II in chloroform (210 ml) at 55° C. was added a solution of bromine (97.17 gm) in chloroform (140 ml) over 80 to 100 min while keeping the temperature between 55 to 60° C. The resulting solution was stirred for about 1 hour. After confirming the completion of reaction on HPLC (starting keto amide should be less than 8%), the mass was cooled to room temperature, and sodium metabisulphite solution (2.8 gm dissolved in 140 ml water) was added, followed by stirring for 5 minutes. The layers were separated and the organic layer was washed with 5% sodium carbonate (7 gm in 140 ml water). The organic layer was washed with water (140 ml) and then concentrated under a reduced pressure at 45-50° C. Hexane (560 ml) was charged and cooled to 0-5° C. and stirred for 1 hour. The separated solids were filtered and the cake was washed with hexane. The solids were dried at 40-45° C. in air oven for 3 to 4 hours until LOD less than 0.5% was obtained to get the title compound.

Yield: 175 gm (91.9%)

Step b) Preparation of Zolpidem Base (Formula V)

2-amino-5-methylpyridine (61.61 gm) of Formula IV (as shown in the accompanied drawing) was added to a stirred solution of N,N-dimethyl-2-bromo-3-(4-methyl)benzoyl propionamide (170 gm) of Formula III in acetone (1190 ml) at 25 to 30° C. The reaction mixture then was refluxed for 18 hours in an oil bath. After cooling the mass to room temperature, the separated solids were filtered and the cake was washed with acetone (2×340 ml). The solids were slurried in demineralised water (850 ml) and the pH of the suspension was adjusted to 6.8 to 7.2 with 10% sodium carbonate solution (200 ml). The mass was stirred at room temperature for 30 minutes and the solids were filtered and washed with demineralised water (2×340 ml) and dried at 45-50° C. under a reduced pressure for 3 to 5 hours to get the title compound.

Yield: 39.5 gm (22.3%)

Step c) Preparation of Zolpidem Hemitartarate (Formula VII)

Zolpidem base (35 gm) of Formula V was dissolved in methanol (140 ml) and to it was added 1.75 gm activated carbon. The resultant mass was stirred at room temperature for 15 minutes and then filtered through hyflobed. A solution of L-(+)-tartaric acid (8.55 gm) of Formula VI dissolved in methanol (70 ml) at 45-50° C. was added to the clear filtrate under stirring. Acetone (280 ml) was added to the reaction mass. The reaction mixture then was seeded with pure zolpidem tartarate (0.2 gm) followed by chilling to −20 to −15° C. The resultant reaction mass was stirred at −20 to −15° C. for a further 2 hours and then the separated solids were filtered. The cake then was washed with acetone (2×70 ml) and then dried at 45 to 50° C. under reduced pressure for 6 to 8 hours to get pure zolpidem hemitartarate of Formula VII.

Yield: 4.2 gm (92.04%)

While several particular forms of the invention have been described, it will be apparent that various modifications and combinations of the invention detailed in the text can be made without departing from the spirit and scope of the invention. For example, the zolpidem hemitartrate synthesized as described above can be formulated using suitable inactive ingredients, as disclosed herein, into tablets or oral dosage forms that are equivalent, for example, to 5 mg and 10 mg of zolpidem tartrate. The resulting tablets can be used for the short-term treatment of insomnia, and to decrease sleep latency and increase the duration of sleep. Accordingly, the invention is not limited, except as by the appended claims.

1. A process for the preparation of zolpidem hemitartarate of Formula VII, 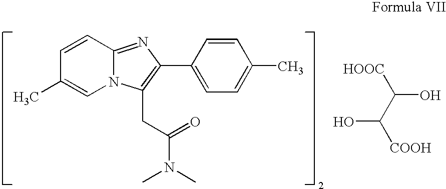
the process steps comprising:
a. reacting N,N-dimethyl-3-(4-methyl)benzoyl propionamide of Formula II 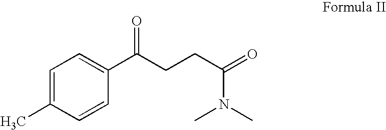
with bromine to get the bromo amide of Formula III; 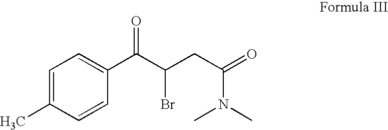
b. condensing the bromo amide of Formula III with 2-amino-5-9 methylpyridine of Formula IV 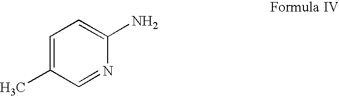
to get the zolpidem base of Formula V; 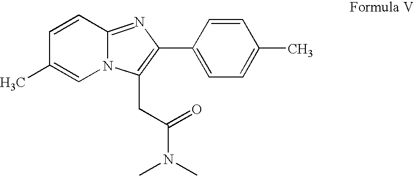
and
c. converting zolpidem base of Formula V to its hemitartarate salt of Formula VII.

2. The process of claim 1, wherein step a) is carried out in an organic solvent.

3. The process of claim 2, wherein the organic solvent is selected from the group consisting of chlorinated hydrocarbon, ether, and mixtures thereof.

4. The process of claim 2, wherein the organic solvent is selected from the group consisting of chloroform, methylene chloride, dichloroethane, dibromoethane, carbon tetrachloride, tetrahydrofuran, diethyl ether, 1,4-dioxane, diisopropyl ether and mixtures thereof.

5. The process of claim 4, wherein the organic solvent is chloroform.

6. The process of claim 1, wherein step b) is performed in the presence of an organic solvent.

7. The process of claim 6, wherein the organic solvent is selected from the group consisting of acetone, diisopropyl ether, dimethylacetamide, dimethylformamide, toluene, methanol, isopropanol, and mixtures thereof.

8. The process of claim 7, wherein the organic solvent is acetone.

9. A pharmaceutical composition comprising the zolpidem hemitartarate of Formula VII of claim 1.

10. The pharmaceutical composition of claim 9, wherein the pharmaceutical composition further comprises one or more pharmaceutically acceptable inactive ingredients.

11. A method of treating insomnia, the method comprising administering a pharmaceutical composition comprising the zolpidem hemitartarate of Formula VII of claim 1.

12. The method of claim 11, wherein the pharmaceutical composition further comprises one or more pharmaceutically acceptable inactive ingredients.
